(19) United States
(12) Patent Application Publication
(10) Pub. No.: US 2023/0389958 A1
(43) Pub. Date: Dec. 7, 2023

(54) TROCAR ASSEMBLY WITH ILLUMINATION AND IMAGING

(71) Applicant: Med Smart Hub Ltd., Caesarea Industrial Park (IL)

(72) Inventor: Moshe Mizrahy, Tel Aviv (IL)

(73) Assignee: Med Smart Hub Ltd., Caesarea Industrial Park (IL)

(21) Appl. No.: 18/247,648

(22) PCT Filed: Oct. 10, 2021
(86) PCT No.: PCT/IL2021/051212

§ 371 Date: Apr. 3, 2023

Related U.S. Application Data
(60) Provisional application No. 63/089,108, filed on Oct. 8, 2020.

(51) Int. Cl. A61B 17/34; A61B 1/05; A61B 1/06

(52) U.S. Cl. CPC A61B 17/3421; A61B 1/0661; A61B 1/05

(57) ABSTRACT

A trocar assembly (10) includes a shaft (12) which extends from a housing (14) and which includes a distal tip (16). An imaging device manipulator (20) is disposed in the housing (14) and coupled to, and operative to move, an imaging device (26) located at a distal portion of the shaft (12). An illumination source (18) is configured to generate light through a light guide (34) towards the imaging device (26).

### FIELD OF THE INVENTION

The present invention relates generally to minimally invasive surgery, and particularly to a trocar assembly with illumination and imaging.

### BACKGROUND OF THE INVENTION

In minimally invasive surgery, there are often several small incisions (such as a primary port and ancillary ports) made into the body to insert surgical tools, insufflation devices, endoscopes, or other viewing devices. There are many advantages in reducing the number of incision points to as few as possible, such as reducing trauma to the patient, reducing the incidence of infection, improving recovery time, and decreasing cosmetic damage. The incisions may be made with a trocar, which is a guide with a sharp tip.

One of the first steps during a laparoscopic surgical procedure involves insufflation of the abdomen with nitrogen or carbon dioxide gas. The resulting expansion of the abdomen reduces the risk of injury to the contents of the abdomen during subsequent insertion of the ports and also allows the surgeons more freedom and space to manipulate instruments and perform the surgery.

Insertion of the primary port is typically accomplished either blindly or through the use of a device that allows some visualization through the laparoscope's camera as the tip of the trocar penetrates the abdominal wall. Insertion of the ancillary ports is generally accomplished while using the laparoscope at the primary port to observe the peritoneum at the ancillary point of insertion and ensure the trocar is not pushed too far into the abdominal cavity.

Laparoscopic surgery is generally performed with only one source of visualization, namely, the camera at the tip of the laparoscope. However, in order to minimize risk of injury to the patient, it is preferable to observe the exit ports of all cannulas every time an instrument is inserted or withdrawn. Such observation currently requires that the camera on the tip of the laparoscope be directed toward a particular port. This would then result in the loss of visualization of the surgical field, which interrupts the surgical procedure and interrupts the use of the surgical instruments until the surgical field can again be visualized with the laparoscope.

Thus an improved trocar visualization device is clearly needed.

### SUMMARY OF THE INVENTION

The present invention seeks to provide an improved trocar assembly with illumination and imaging, as is described more in detail hereinbelow.

There is thus provided in accordance with an embodiment of the present invention a trocar assembly including a shaft which extends from a housing and which includes a distal tip, an imaging device manipulator disposed in the housing and coupled to, and operative to move, an imaging device located at a distal portion of the shaft, and an illumination source configured to generate light through a light guide towards the imaging device.

The imaging device manipulator may be coupled to the imaging device through a linkage shaft. The imaging device may be pivoted to the linkage shaft.

The imaging device may be rotatable about a longitudinal axis of the shaft, and may be pivotable with respect to the longitudinal axis of the shaft.

The imaging device may be movable in translation.

The light guide may be a wall thickness of the shaft. The distal end of the light guide may be beveled.

### BRIEF DESCRIPTION OF THE DRAWINGS

The present invention will be understood and appreciated more fully from the following detailed description, taken in conjunction with the drawing in which:

FIG. 1 is a simplified illustration of a trocar assembly, constructed and operative in accordance with a non-limiting embodiment of the present invention;

FIG. 2 is an enlarged illustration of a portion of the trocar assembly of FIG. 1, showing a trocar seal, light source and light guide;

FIG. 3 is an enlarged illustration of a portion of the trocar assembly of FIG. 1, showing the light guide and an imaging device, such as a camera; and

FIGS. 4 and 5 are illustrations of different fields of view of the imaging device.

### DETAILED DESCRIPTION

Reference is now made to FIG. 1, which illustrates a trocar assembly 10, constructed and operative in accordance with a non-limiting embodiment of the present invention.

The trocar assembly 10 includes a shaft 12, which extends from a housing 14. Shaft 12 may extend perpendicularly from housing 14, or at other non-perpendicular angles. Shaft 12 may include a distal tip 16, which may be either a pointed cutting blade or a blunt tip, so that the trocar assembly 10 may be used to puncture skin or enter through a separately made incision.

Different components may be disposed inside housing 14, such as but not limited to, an illumination source 18, such as one or more LEDs or any other type of light, an imaging device manipulator 20, control electronics 22, and communication components 24, such as a transmitter and/or receiver for wired or wireless communication with a computer, personal communication device, cloud-based data and control, etc.

In one non-limiting embodiment, the imaging device manipulator 20 includes a remote-controlled servomotor or actuator coupled to an imaging device 26, such as through a linkage shaft 28. The imaging device 26 may be a camera, ultrasound sensor or other suitable imaging modality sensor. The imaging device 26 may be mounted on an arm 30 which is pivotally coupled to linkage shaft 28. The imaging device manipulator 20 is coupled to both linkage shaft 28 and arm 30. In this manner, imaging device manipulator 20 can rotate linkage shaft 28, thereby rotating imaging device 26 completely (360° and more) about the longitudinal axis of shaft 12, and can also pivot (tilt) imaging device 26 with respect to the longitudinal axis of shaft 12, such as, without limitation, in a range of plus/minus 100° or more. Thus, imaging device 26 is coupled at a swivel joint or gimbal to linkage shaft 28 and can capture images at multiple degrees of freedom. Optionally, the imaging device 26 can be moved in translation, too.

The housing 14 may be affixed to the patient skin with adhesive or other fastening means.

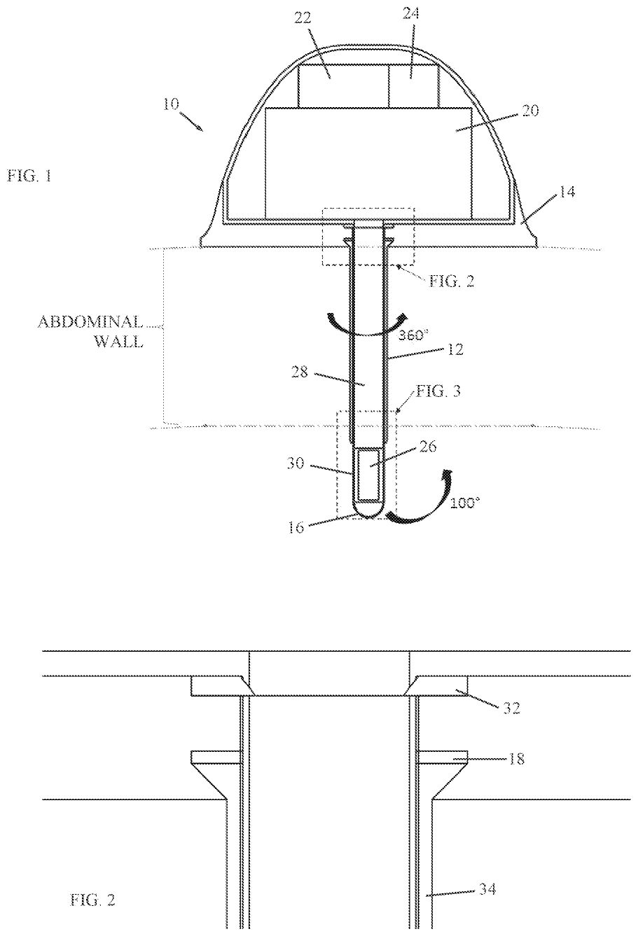

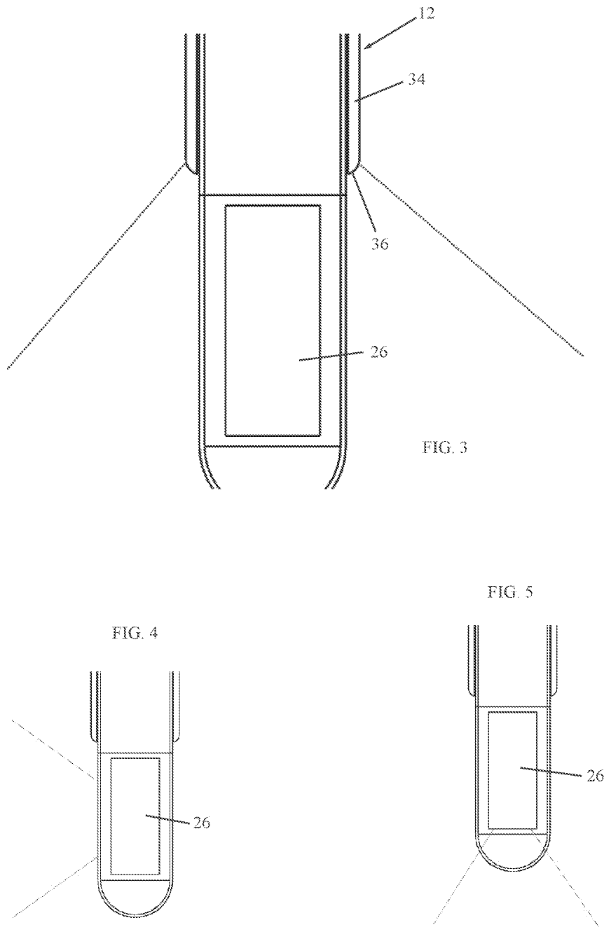

Reference is now made to FIG. 2. The trocar assembly 10 may include a seal 32 that seals the inner components from bodily fluids and other contaminants. The illumination source 18 may generate light which is directed through a light guide 34, which may be the wall thickness of shaft 12. Shaft 12 may be made from an optically transparent material, such as but not limited to, poly methyl methacrylate (PMMA). As seen in FIG. 3, the distal end 36 of light guide 34 may be beveled to disperse the lighting at any desired angle. The beveled distal end may serve as the sharp end of shaft 12 (distal tip 16) for puncturing skin.

Reference is now made to FIGS. 4 and 5. The imaging device 26 may have a side-looking field of view (FIG. 4) or a forward-looking field of view (FIG. 5). As mentioned before, the imaging device 26 can capture images at multiple degrees of freedom and multiple fields of view.

What is claimed is:

1. A trocar assembly (10) comprising:
a shaft (12) which extends from a housing (14) and which comprises a distal tip (16);
an imaging device manipulator (20) disposed in said housing (14) and coupled to, and operative to move, an imaging device (26) located at a distal portion of said shaft (12); and
an illumination source (18) configured to generate light through a light guide (34) towards said imaging device (26).

2. The trocar assembly (10) according to claim 1, wherein said imaging device manipulator (20) is coupled to said imaging device (26) through a linkage shaft (28).

3. The trocar assembly (10) according to claim 2, wherein said imaging device (26) is pivoted to said linkage shaft (28).

4. The trocar assembly (10) according to claim 1, wherein said imaging device (26) is rotatable about a longitudinal axis of said shaft (12), and is pivotable with respect to the longitudinal axis of said shaft (12).

5. The trocar assembly (10) according to claim 1, wherein said imaging device (26) is movable in translation.

6. The trocar assembly (10) according to claim 1, wherein said light guide (34) comprises a wall thickness of said shaft (12).

7. The trocar assembly (10) according to claim 6, wherein a distal end (36) of said light guide (34) is beveled.

8. The trocar assembly (10) according to claim 1, wherein said imaging device (26) has a side-looking field of view.

9. The trocar assembly (10) according to claim 1, wherein said imaging device (26) has a forward-looking field of view.

10. The trocar assembly (10) according to claim 1, further comprising communication components (24).
